(19) United States
(12) Patent Application Publication
(10) Pub. No.: US 2011/0220120 A1
(43) Pub. Date: Sep. 15, 2011

(54) Method and Apparatus For Occluding A Lumen

(76) Inventors: John R. Frigstad, St. Anthony, MN (US); Paul W. Danielson, Shakopee, MN (US); Kurt Krueger, Stacy, MN (US); Steven Palter, Sea Cliff, NY (US); Ov Slayden, Portland, OR (US)

(21) Appl. No.: 13/113,909

(22) Filed: May 23, 2011

Related U.S. Application Data
(63) Continuation of application No. 11/871,684, filed on Oct. 12, 2007, now abandoned.
(60) Provisional application No. 60/829,206, filed on Oct. 12, 2006.

(51) Int. Cl. A61F 6/06

(52) U.S. Cl. 128/830

(57) ABSTRACT

A method and apparatus of occluding a lumen of a body cavity, more preferably, the lower uterine segment and/or cervix of a female patient is provided that results in a significant reduction or complete elimination of uterine bleeding, thereby providing a treatment option for conditions such as menorrhagia or dysmenorrhea.

### RELATED APPLICATIONS

This application claims priority benefit from U.S. Provisional Application Ser. No. 60/829,206, filed Oct. 12, 2006 entitled Method And Apparatus For Occluding A Lumen, the entirety of which is incorporated by reference herein.

### BACKGROUND OF THE INVENTION

Menstrual bleeding is a part of normal life for women. The onset of menstruation, termed menarche, usually occurs at the age of 12 or 13. The length of a woman's monthly cycle may be irregular during the first one to two years. Once the menstrual cycle stabilizes, a normal cycle may range from 20 to 40 days, with 28 days commonly being an average. Age, weight, athletic activity and alcohol consumption are several factors that affect menstrual cycles. For example, younger women (under the age of 21) and older women (over the age of 49) tend to have longer cycle times, generally averaging 31 days and over. Similarly, women who are very thin or athletic also have longer cycles. In contrast, women who consume alcohol on a regular basis tend to have shorter cycle times.

Nearly all women, at some time during their reproductive life, experience some type of menstrual disorder. These disorders range from mild to severe, often resulting in numerous lost work hours and the disruption of personal/family life each month. In general, physical symptoms such as bloating, breast tenderness, severe cramping (dysmenorrhea) and slight, temporary weight gain frequently occur during most menstrual cycles. In addition to physical symptoms, emotional hypersensitivity is also very common. Women report a wide range of emotional symptoms, including depression, anxiety, anger, tension and irritability. These symptoms are worse a week or so before a woman's menstrual period, generally resolving afterward.

Many women also suffer from a condition called menorrhagia (heavy bleeding). Menorrhagia is a clinical problem characterized by extremely heavy flow/bleeding and major discomfort characterized by blood loss exceeding 80 cc/month. It is estimated that 1 in 5 women between the ages of 35 and 50, or approximately 6.4 million women in the United States alone, are affected by menorrhagia. Fibroids, hormonal imbalance and certain drugs, such as anticoagulants and anti-inflammatory medications, are common causes of heavy bleeding.

Women diagnosed with menorrhagia or dysmenorrhea have limited treatment options available to them. Currently, other than hormone therapy and a few experimental pain management techniques, hysterectomy (removal of the uterus) and endometrial ablation/resection (destruction of the lining of the uterus) are the clinically accepted treatment modalities for menorrhagia. Both of these surgical procedures eliminate the possibility of childbearing. Further, hysterectomy requires up to a six week recovery time and a lifetime of hormone therapy when the ovaries are removed. Endometrial ablation has a low success rate at achieving amenorrhea (cessation of menstrual bleeding). As a result, many of the women affected by menorrhagia are driven to make lifestyle-altering decisions.

Over 600,000 hysterectomies are performed each year in the United States. It is estimated that 1 in 3 women in the U.S. have a hysterectomy before the age of 65. Menorrhagia is the most common reason why hysterectomies are performed. Several studies have estimated that menorrhagia is the cause of 30% (some studies as high as 50%) of the 600,000 annual hysterectomies, resulting in a basis of 180,000 to 300,000 procedures annually. Financially, these numbers translate into annual hospital costs that exceed $5 billion per year.

Based on these statistics, hysterectomy is a very common operation. In general, there are three types of hysterectomies: partial, total and radical. A partial hysterectomy involves removal of the upper portion of the uterus, leaving the cervix and the base of the uterus intact. A total hysterectomy involves removal of the entire uterus and cervix. A radical hysterectomy entails removal of the uterus, both Fallopian tubes, both ovaries, and the upper part of the vagina. Each of the above three procedures may be performed via an abdominal incision (abdominal hysterectomy) or through a vaginal incision (vaginal hysterectomy).

After the operation, the hospital stay is generally less than a week, depending on the type of hysterectomy and whether there are any complications. Since a hysterectomy is a major operation, discomfort and pain from the surgical incision are most pronounced during the first few days after surgery. Medication is available to minimize these symptoms. By the second or third day, most patients are up walking. Normal activity can usually be resumed in four to eight weeks and sexual activity can usually be resumed in six to eight weeks.

Since the 1800's, attempts using various treatments have been made to control uterine bleeding by means other than hysterectomy. Alternative methods include chemicals, steam, ionizing radiation, lasers, electrocautery, cryosurgery and others. The long-term risk for such procedures is quite high and may lead to other more serious complications such as mixed mesodermal tumors or uterine cancer.

Typical therapy or treatment options include drug therapy, followed by dilation and curettage (D & C), endometrial ablation, and, as a last resort, hysterectomy. Drug therapy is generally the first treatment option employed to treat excessive bleeding. Birth control pills, progestin, danazol and gonadotropin-releaseing hormone (GnRH) are a few examples of drug treatments prescribed to reduce bleeding. In general, birth control pills contain synthetic forms of estrogen and progesterone, which prevent ovulation and, thereby, reduce endometrial build-up or thickness. As a result, pill users normally have lighter or minimal menstrual bleeding. Progestin, another synthetic form of progesterone, balances the effects of estrogen normally produced by the body and, similar to the pill, reduces endometrial growth. Often, Danazol and other GnRH agents are prescribed to suppress estrogen production and ovulation. As a result, menstrual bleeding stops or is significantly reduced. However, side-effects of such treatments may include bloating, breast tenderness, increased risk of osteoporosis and high cholesterol.

D & C, frequently a second treatment option for excessive bleeding, is a very common, minor surgical procedure that is generally performed on an outpatient basis in a hospital. Usually, the patient is given a general anesthetic, although the procedure occasionally is performed using only a local anesthetic. The dilation step of the procedure involves dilating or stretching the cervix, which is the lower part of the uterus. Once the cervix is appropriately dilated, the curettage step can then be performed. During curettage, a curette (a spoon-shaped instrument) is inserted through the vagina, past the cervix and into the uterus. The curette is then used to scrape and/or collect tissue from the inside surfaces of the uterus.

Endometrial ablation has become more popular and has been offered as another alternative treatment to hysterectomy for patients suffering from menorrhagia. In 1996, 179,000 ablation procedures were performed, up from 49,000 in 1993. This technique is intended to permanently ablate all layers of the endometrium and allow the cavity to become lined with fibrous tissue.

In general, endometrial ablation is less costly and requires less recovery time for the patient. However, the procedure has received mixed results for controlling bleeding, depending on the technique used, and has a limited success rate of no greater than 20% when defined as complete cessation of bleeding. During one five-year study of 525 women with an average age of 42, endometrial ablation completely stopped uterine bleeding only 26% to 40% of the time. However, approximately 79% to 87% of the women said they were satisfied with the surgery. About 16% of the women required a repeat ablation to stop bleeding and 9% of the women ultimately opted for a hysterectomy. Research has also shown that the effectiveness of endometrial ablation may decline over years, with menstruation returning in about one-third of women.

It should be noted, however, that the goal of endometrial ablation was never to create amenorrhea (cessation of menstrual periods). This procedure was originally developed as a less invasive alternative to hysterectomy in order to return women with menorrhagia to a normal menstrual flow.

In either endometrial ablation or resection, an attempt is made to remove or destroy the entire lining of the uterus (the endometrium). Endometrial resection, first described in 1983 by De Cherney et al., involves the use of a resectoscope-cutting loop to perform endometrial ablation to remove the lining of the uterus. In contrast, ablation generally uses either vaporization, coagulation or some other thermal energy source to destroy the uterine lining.

In 1894, Heinrich Fritsch was the first to describe amenorrhea resulting from traumatic obliteration of the uterine cavity following puerperal curettage. However, it was not until 1948, that knowledge about uterine adhesions was first disseminated in medical journals by Joseph G. Asherman, for whom the condition is named. In 1957, the 17^{th} Congress of the Federation of French Speaking Societies of Gynecology and Obstetrics proposed the following classification of uterine synechiae: Traumatic Synechiae connected with surgical or obstetrical evacuation of the uterus; Spontaneous synechiae of tuberculosis origin; Synechiae occurring after myomectomy; and Synechiae secondary to the attack of chemical or physical agents and likewise those resulting from atrophic changes.

In general, two types of traumatic synechiae are currently recognized. The first type is stenosis or obliteration of the cervical canal. The second type of traumatic synechiae is partial or complete obliteration of the uterine cavity by conglutination of the opposing walls.

Other terms, such as endometrial sclerosis, traumatic uterine atrophy, uterine atresia, uterine synechiae and adhesive endometriosis, have also been used to describe the phenomena of Asherman's Syndrome. The severity of adhesion is generally classified into one of the following three groups or classes: Class I represents adhesions occurring in less than one-third of the uterine cavity with both ostia (i.e. openings of the Fallopian tubes) visible; Class II represents adhesions occurring in one-third to one-half of the uterine cavity with one ostium visible; and Class III represents adhesions occurring in greater than one-half of the uterine cavity with no ostia visible.

Although Asherman's Syndrome has been studied extensively and numerous articles and papers have been written on the topic, uncertainty still exists as to the predominant causative factor(s) and biological mechanism(s). It is believed that if the endometrium is severely damaged, it may be replaced by granulation tissue. When this happens, the opposing uterine walls adhere to one another and form scar tissue. In particular, adhesions form and transluminally bridge the anterior and posterior surfaces of the uterus. The adhesions or tissue formed between the walls comprises connective tissue that is, typically, avascular. Soon after, the tissue may be infiltrated by myometrial cells and, later, covered by endometrium. As a result of this tissue transformation, many patients encounter a significant decrease or elimination of menstrual bleeding. Hence, there is a need to develop a safe way to apply the mechanics of Asherman's syndrome to the patients suffering from menorrhagia in order to reduce or eliminate bleeding without the risks associated with other treatments. Developments have begun toward achieving positive results in this direction. Abnormal uterine bleeding treatments incorporating tissue transformation mechanics have been described in U.S. Pat. No. 6,708,056 to Duchon et al., and U.S. Provisional Application No. 60/762,333 entitled Apparatus and Method of Resetting a Uterine Cavity, filed Jan. 25, 2006, both of which are herein incorporated by reference in their entireties.

In view of the above, there is a need for a minimally invasive device and method to treat abnormal intrauterine bleeding. In particular, it is desirable that the device have a high success rate at treating menorrhagia and have minimal to no side effects or related complications. Such a device must also be biocompatible and non-toxic. In addition, the related treatment methods should reduce patient recovery times and hospital costs. Overall, the method of treatment should also improve the quality of life for patients.

### BRIEF SUMMARY OF THE INVENTION

The present invention relates to devices and methods for correcting or regenerating dysfunctional human tissue. In particular, the invention relates to devices and methods for treating dysfunctional uterine conditions in women. More specifically, the present invention relates to dysfunction of the endometrial or myometrial layers of a uterus. The present invention further relates to establishing a mechanism of action by which identified dysfunctional uterine tissues may thereafter be treated, corrected or reversed.

In general, the present invention contemplates an implantable device for treating excessive bleeding in a body cavity. The device comprises a biocompatible material deliverable into a body cavity. The device further comprises a configuration that at least partially occludes a lumen of a body cavity. Additionally, such device may further comprise a configuration that adheres with surrounding body cavity and/or lumen tissue. The device may also comprise a configuration that is comprised of at least partially curable, polymeric components.

The present invention also contemplates a method of occluding a lumen of a body cavity. In general, the method comprises inserting an implantable device at least partially within a lumen of a body cavity and/or a body cavity. The method also includes placing the implantable device at an optimal site within a lumen and/or a body cavity, wherein the optimal site promotes treatment and/or cessation of bleeding, and more specifically, uterine bleeding. The present invention also comprises one or more methods for preparing a tissue bed to accept implantable material.

### BRIEF DESCRIPTION OF THE DRAWINGS

Other features and advantages of the present invention will be seen as the following description of particular embodiments in conjunction with the drawings, in which:

FIG. 1 is a front view of an exemplary uterus of a female patient as described in accordance with the present invention.

FIG. 2 is a front view of one embodiment of an implant as placed in a female patient according to the present invention.

FIG. 3 is a front view of one embodiment of an implant as placed in a female patient according to the present invention.

FIG. 4 is a perspective view of an embodiment of an implant according to the present invention.

FIG. 5 is a perspective view of an embodiment of an implant according to the present invention.

FIG. 6 is a perspective view of an embodiment of an implant according to the present invention.

FIGS. 7A and B are cutaway views of an embodiment of an implant according to the present invention.

FIG. 8A and B are cutaway views of an embodiment of an implant according to the present invention.

FIG. 9 is a perspective view of an embodiment of an implant according to the present invention implanted in a lumen.

FIG. 10 is a perspective view of an embodiment of an implant according to the present invention.

FIG. 11 is a perspective view of an embodiment of an implant according to the present invention.

FIG. 12 is a perspective view of an embodiment of an implant according to the present invention.

FIG. 13 is a perspective view of an embodiment of an implant according to the present invention.

FIG. 14 illustrates an embodiment of an implant according to the present invention implanted in a cervix

FIGS. 15A and 15B illustrate an embodiment of an implant according to the present invention implanted around a cervix.

FIGS. 16A-C illustrates an embodiment of delivery system being used to deliver an implant according to the present invention.

### DETAILED DESCRIPTION OF THE PREFERRED EMBODIMENT

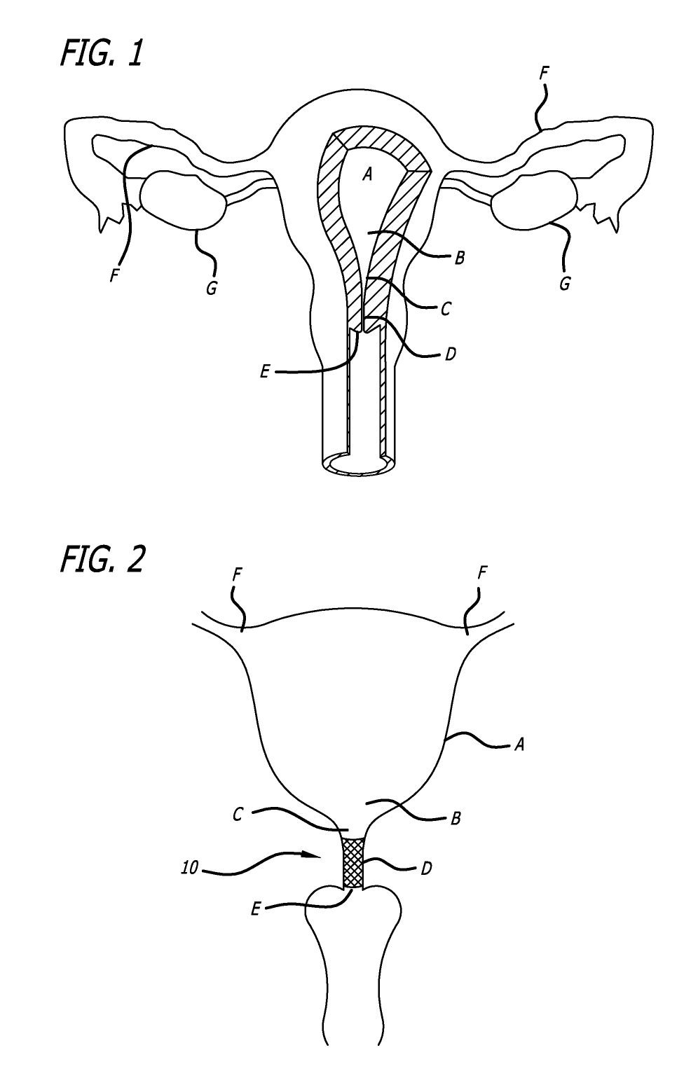

FIG. 1 is an illustration of the female reproductive system, showing a uterus A, a cervix D and fallopian tubes F. The juncture between the uterus A and cervix D is often referred to in the art as the lower uterine segment (LUS) B. The cervix D includes an internal cervical orifice (internal cervical os) C and an external cervical orifice (external cervical os) E. Generally, the internal cervical os C measures approximately 2.3 mm to 6 mm in width, depending upon factors such as parity, the selected method of measurement and the day of a female patient's menstrual cycle when the measurement is made. The length of the internal cervical os C is approximately 10 mm. Also illustrated are the fallopian tubes F and ovaries G.

The methods and devices of the present invention are directed to occluding the passage of blood through the cervix. In some embodiments, the occlusion is a complete blockage; in others, blood flow is merely restricted. The result in either case is a reduction or elimination of the body's production of menstrual blood.

Although it is not fully understood why occluding the lumen of a body cavity creates the following proposed response, nor does the inventor want to be bound by the proposed theory, it is believed by the inventor that the blood remaining in the cavity results in one or more responses, including (but not limited to), for example, an increase in cavity pressure and/or a chemical and/or biological response (e.g., resulting from the presence and/or absence of hormones and/or proteins in the retained blood). It is additionally hypothesized that pooling of blood in the uterus (as a result of the herein-described occlusion) directly affects the state of the endometrium. For example, one theory is that proteins, such as matrix metalloproteinases (MMPs), which are present in menstrual blood, “eat” fibronectin, a substance which is necessary for the rebuilding of the endometrium. Thus, the endometrium appears attenuated or shorter.

Such responses are thought to create a change in peristaltic contractile wave patterns and a neuromuscular feedback response. It is hypothesized that this response mimics what would be seen in an Asherman's Syndrome patient, who has an adhesion causing an occlusion in the LUS or cervix and clinically presents with reduced or eliminated periods.

In at least one embodiment of the present invention, these responses or combination thereof, may cause the endometrium to become attenuated. In such instances, the endometrium may visually appear to be normal but shorter than expected, or appears to be out of phase, and/or becomes dysfunctional, resulting in greatly reduced flow or amenorrhea.

FIG. 2 illustrates a general embodiment of an implant or occluding device 10 placed in a female patient according to the present invention. The implant 10 is designed to block or occlude a lumen leading to or from a body cavity, such as the uterus. The implant 10 is shown placed in the cervix D; however, it is also contemplated that the implant 10 may also or alternatively be placed in the LUS B and/or one or both of the fallopian tubes F. It is preferred that the implant 10 be placed in a lumen in which the lumen tissue has been pre-treated both to receive the implant 10 and facilitate ingrowth of the tissue into the implant 10. The phenomena of tissue ingrowth into an implant 10 is described in greater detail in U.S. Patent Publication No. 2005/0031662 to Danielson et al., which is herein incorporated by reference in its entirety.

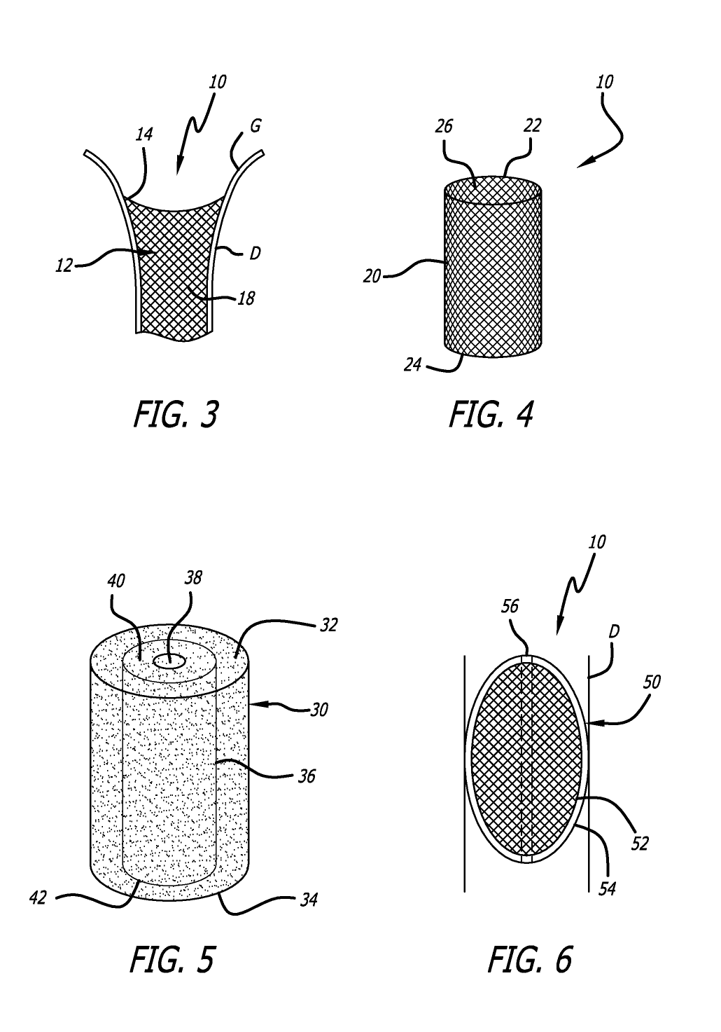

Several embodiments of the implant 10 are contemplated for use with the present invention and shown and described herein. Generally, the various embodiments of the implant 10 have somewhat circular or oval cross-sections and may be/are somewhat porous to promote ingrowth. More details on individual embodiments of the implant 10 are attained by turning to the figures. The various materials and general mechanical principals that apply to the various embodiments of the implant 10 are included in the description of a basic design for the implant 10 shown in FIG. 3. However, it is understood that FIG. 3 is being used to provide general background about the various implants 10, in addition to being representative of a specific embodiment. Thus, the various features of the implant 10 included in the description of FIG. 3 are intended to be applicable to all of the embodiments shown in the Figures and described herein. For that matter, one skilled in the art will contemplate that any of the unique features shown in any of the Figures may find application in combination with any of the other features shown in any of the other Figures and/or described herein.

Turning now to FIG. 3, a first embodiment of the implant 10 is shown. The implant 10 of FIG. 3 is generally a flexible cylinder having a body 12, an upper end 14. The body 12 is preferably a mesh or foam material that promotes ingrowth. If mesh, the mesh may be braided, woven, non-woven, fenestrated, knitted, or formed in any other manner. The mesh may be formed of one continuous strand or a plurality of strands 18. The strands 18 may be a wire formed of a resilient, biocompatible metal, such as stainless steel or Nitinol or any other suitable biocompatible material, such as a polymer, nylon, silicone, ePTFE, polyester fiber (e.g., Dacron), polyethylene etc. If the body 12 is made of foam, any one of the materials or any combination of these materials may be used.

The body 12 is preferably designed to promote ingrowth. For example, the foam density, or the materials and the intertwining of the strands 18 to form the mesh, preferably result in a degree of porosity that facilitates optimal uterine and/or cervical tissue ingrowth into the implant 10. At a minimum, it is preferred that the porosity size of an implant is within the range of 300-600 microns and most preferably, centered on about 400 microns. Further information regarding desired dimensions and the resulting performance and functionality of porosity are described in detail in U.S. Pat. Nos. 5,605,693, 5,589,176, 5,681,572 and 5,624,674 to Seare, Jr., which are herein incorporated by reference in their entireties.

The body 12 of implant 10 may be comprised of an inert foam material shaped accordingly to match the geometry of the LUS and/or cervix. The inert foam material preferably has pores of sufficient size to allow for tissue ingrowth and vascularization of that ingrowth. The porosity of the foam material may exhibit interconnected porosity throughout the entire volume of the implant 10 or interconnected porosity in at least one layer surrounding a solid core of the same, similar or different core material. Some suggested materials for use with the implant 10 allowing for tissue ingrowth may include silicone, ePTFE, polyester fiber (e.g., Dacron), polyethylene and others. Embodiments of the implant 10 with such features are described in more detail below.

The body 12 of implant 10 may be bioabsorbable. The desired tissue changes are likely permanent and, after the changes have been established, there is no longer a need for the implant 10. However, mechanical removal of the implant would be difficult due to the ingrowth. Hence, a bioabsorbable material is a logical choice. The bioabsorbable material could be any one of a number of materials known to those of skill in the art. Examples of bioabsorbable materials contemplated for use in the present invention are described in detail in U.S. Pat. No. 6,514,515, which is herein incorporated by reference in its entirety.

In addition to mesh or foam, the body 12 is also contemplated as being an injectable polymer, used either alone or in combination with one of the body materials. The injectable polymer may be curable either in vivo or in vitro, depending on the desired effect and placement method. The injectable polymer preferably has the capability to adhere to the surrounding body cavity tissue. The porosity of the cured, injectable polymer may be either configured to allow for some fluid passage or provide a total restriction of fluid passage from the uterus.

Additionally, as shown in FIG. 3, the upper end 14 of the implant 10 in this embodiment is somewhat concave and the body 12 is generally cylindrical. It is believed by the inventor a concave upper end 14 of the implant 10 at the tissue wall G—implant 10 interface may provide an optimal angle and/or smoother transition between the tissue wall G and the implant 10 that will help promote optimal tissue ingrowth (e.g., myoometrial tissue, etc.) and prevent undesirable tissue ingrowth (e.g. endometrial tissue, etc.) into the implant 10. The mesh body 12 of the implant 10 may also extend throughout the center of the implant, thereby creating a solid mesh plug, or may define a small lumen through the implant 10.

For example, FIG. 4 shows in implant 10 that has a tubular mesh body 20 that forms a cylinder with an upper end 22, a lower end 24, and a lumen 26 extending therebetween. The mesh body 20 is formed as described above in the description of the implant 10 of FIG. 3, and preferably similarly promotes ingrowth. The upper end 22 of the embodiment of FIG. 4 is relatively straight.

The porous nature of the embodiment of the implant 10 of FIG. 4, including the existence of the lumen 26 makes the implant 10 extremely flexible. The flexibility of the implant 10 enhances maneuverability and eases deployment. The implant 10 may be self-expanding or mechanically expandable.

FIG. 5 illustrates an implant 10 having a cylindrical body 30, a closed upper end 32 and a closed lower end 34. The implant 10 also includes a solid core 36 suspended within the porous surrounding material of the body 30. The solid core 36 of the implant 10 may optionally include an axial lumen 38 for access through the implant 10 and/or release of fluids from the uterus. The axial lumen 38 may extend through the porous material at the ends 32 and 34 of the body 30 or, as shown, the closed ends 32 and 34 may extend over the ends 40 and 42 of the core 36, respectively. Providing porous body material over the ends of the lumen 38 may be used as a means for controlling the flow rate through the lumen 38, to ensure a degree of pooling occurs in the uterus. Alternatively, the diameter of the lumen 38 can be designed to control the flow rate therethrough. The core 40 may also extend longitudinally such that its ends 40 and 42 are flush with the ends 32 and 34 of the body 30.

It is contemplated that the solid core 36 provides stability to the otherwise porous implant 10 and thereby prevents migration until ingrowth is established. Additionally, limiting the depth of the porous material may promote ingrowth. For example, there may be a better likelihood of optimal tissue ingrowth when the tissue only has a distance of 1 mm to migrate into the implant 10 versus if the tissue has a distance of 3 mm to migrate into the implant 10. The solid core 36 may also act to provide pressure on the wall of the cervix, pushing the porous material into the tissue wall, further catalyzing the ingrowth process. It is also believed that an implant 10 having a thin porous body 30 limited in depth by a solid core 36 may reduce the chance of infection.

The solid core 36 may be formed of biocompatible metal, such as stainless steel or Nitinol or any other suitable biocompatible material, such as a polymer, nylon, silicone, ePTFE, polyester fiber (e.g., Dacron), polyethylene etc.

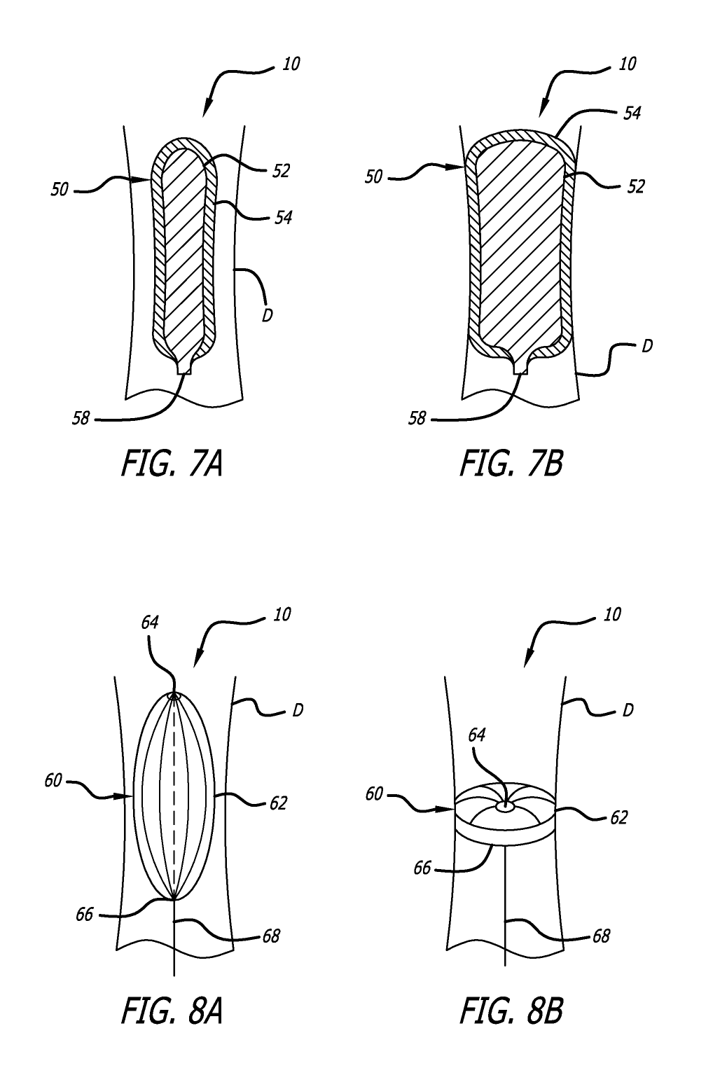

FIGS. 6 and 7 show an implant 10 having a body 50 that is formed by a balloon 52 surrounded by a porous layer 54. This embodiment provides the advantages of the solid core of the embodiment of FIG. 5 but provides the additional advantages of insertion ease and pressure control. During the implantation of this embodiment of implant 10, the physician places the implant 10 in the cervix or other body lumen in a deflated state, as shown in FIG. 7a, and then inflates the balloon 52 to a desired size and pressure, as seen in FIG. 7b. The inflatable balloon core 52 not only conforms to the shape of the lumen, but places a controlled amount of radial pressure on the lumen wall. Controlling the amount of pressure placed on the lumen wall also controls the thickness of the porous covering 54. This gives the physician the ability to optimize ingrowth.

The balloon 52, when used in the cervix D, may be inflated with air or some other inflating media, such as saline or a biocompatible gel polymer, depending upon the desired effect. Air may be more susceptible to permeation than thicker media. The body 50 may be optionally formed with a lumen 56 extending therethrough through which fluids may be released from or delivered into the uterus. The balloon 52 preferably includes a self-sealing port 58 in order to ease the inflation procedure.

FIGS. 8A and 8B show another embodiment of an implant 10 having a body 60 that includes a shaped balloon 62 with an upper end 64 and a lower end 66. The balloon 62 also includes a cinch cord 68 running through the balloon that is attached to the upper end 64. Once the balloon 62 is inserted in a deflated state (FIG. 8A), the balloon 62 is inflated and the cinch cord is pulled tight, drawing the upper end 64 and the lower end 66 together to reshape the body 60 into a torus-shaped implant 10 as shown in FIG. 8B. The body 60 may also be covered with a porous covering to promote ingrowth, though not shown in order to show the detail of the balloon 62. The act of pulling the ends together provides radial pressure against the walls of the cervix. It is contemplated that the balloon 62 be inserted in a partially inflated state, as the volume of the balloon 62 is reduced during the cinching process, obviating the need for further inflation once inserted.

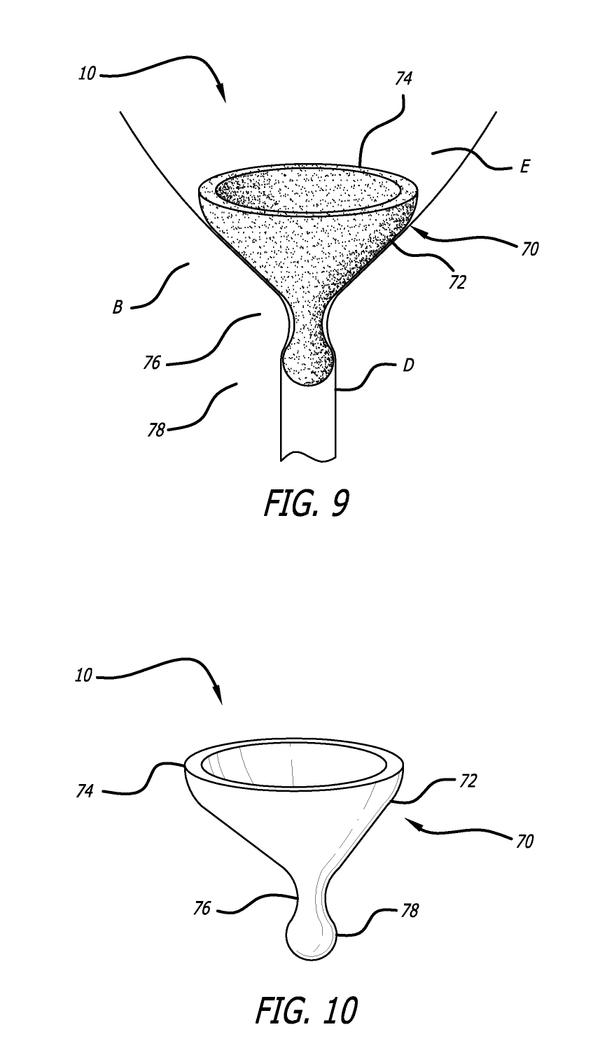

FIGS. 9 and 10 show an implant 10 having a body 70 that includes a conical portion 72 having a wide upper end 74 and a narrow lower end 76, and a bulbous portion 78 below the lower end of the conical portion 72. The conical portion 72 is shaped to conform to the LUS B and the bulbous portion 78 is shaped to anchor the implant 10 in the cervix D. The wide upper end 74 may be either open or closed, resulting in a body 70 that is either substantially hollow or substantially filled, respectively. The body 70 is either porous as shown in FIG. 9 and as described above in the description of FIG. 3, or the body 70 may be solid, as shown in FIG. 10.

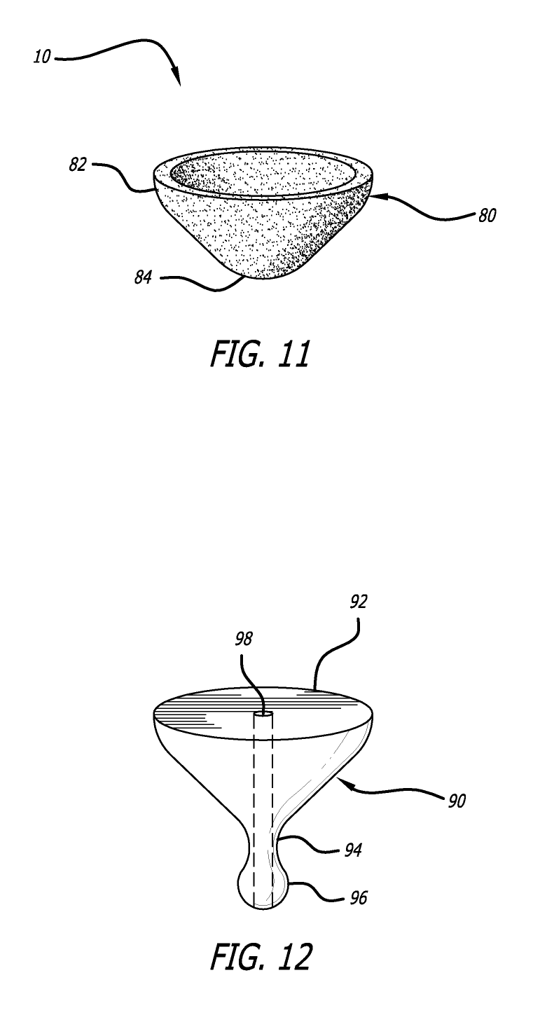

FIG. 11 illustrates a implant 10 having a body 80 that is substantially conical in shape, having a wide upper end 82 and a narrow lower end 84. As with the implant of FIG. 9, the body 80 is shown as having an open upper end 82 and a closed lower end 84. However, the upper end 82 may be closed, resulting in a substantially solid, or at least homogenously porous, body 80.

FIG. 12 illustrates another conical embodiment of an implant 10 having a conical body 90 with a wide upper end 92 and a narrow lower end 94 with a bulbous portion 96 below the narrow lower end 94. The upper end 92 and the lower end 94 are closed. The otherwise homogenous body 90 has a lumen 98 passing therethrough for the release of fluids from the uterus.

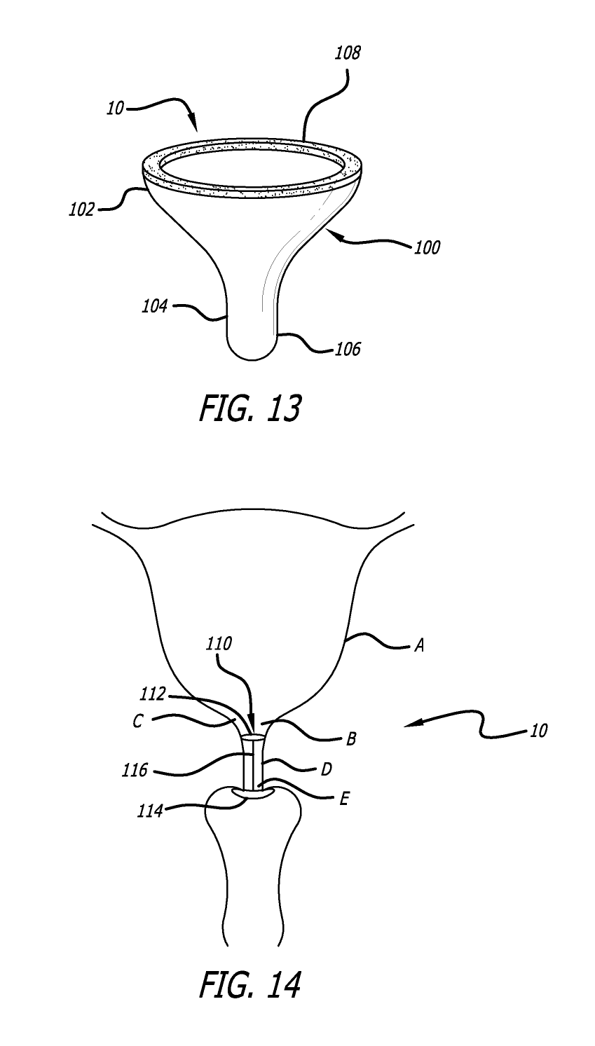

FIG. 13 illustrates a implant 10 (similar to FIGS. 7, 10 and 11) having a a body 100 that is substantially conical in shape, having a wide upper end 102 and a narrow lower end 104 and a tip portion 106 below the narrow lower end 104. The wide upper end 102 is substantially open, creating a body 100 that is substantially concave. The body 100 is mostly solid but includes a porous portion 108 around the upper end 102 to promote peripheral ingrowth therearound.

FIG. 14 illustrates an implant 10 having a body 110 that includes an upper portion 112 and a lower portion 114 connected together by a tether 116. The upper portion 112 is sized to nest in the LUS B and block the internal cervical os C, while the lower portion 114 is shaped to surround the external cervix os E. The tether 116 pulls the two portions 112 and 114 together, locking the body 110 in place. A lumen (not shown) may be provided through the upper and lower portions 112 and 114 to provide a controlled amount of drainage from the uterus. Either of the portions 112 and 114 may be solid or porous as described above.

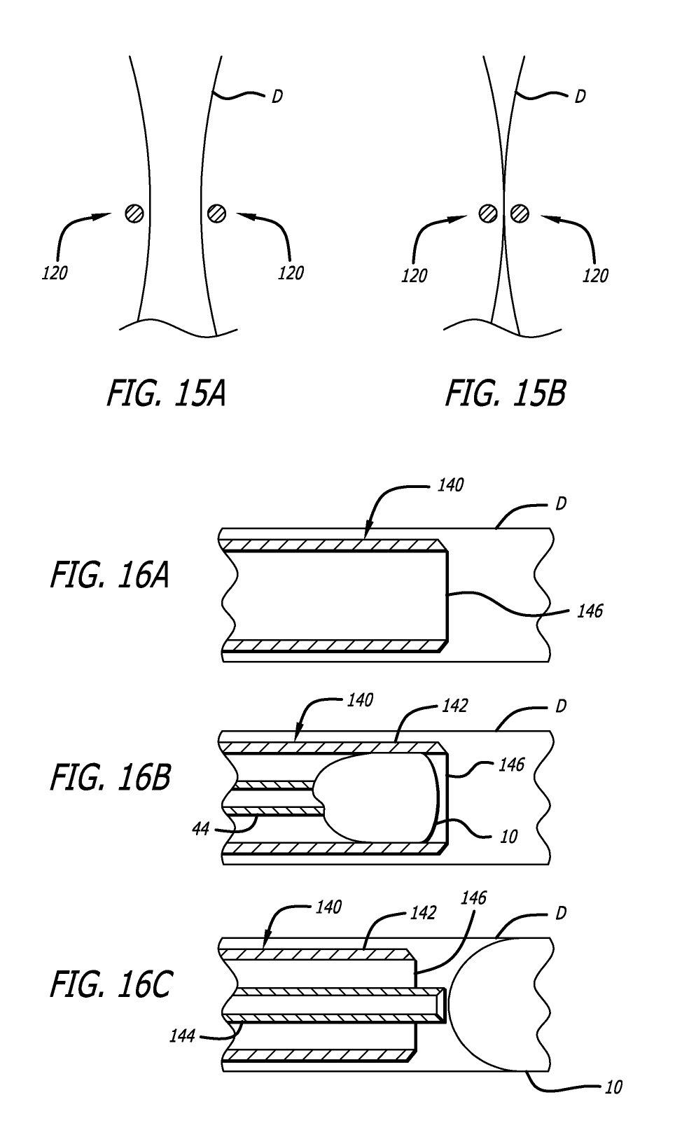

FIGS. 15A and 15B illustrate an implant 120 that is formed by injecting a bulking material into the tissue surround the cervix D. “Bulking” means injecting or placing materials in the body tissue surrounding a lumen of a body cavity in a manner sufficient to constrict the outer region of a lumen, thereby narrowing and thus occluding the lumen. As shown in the Figures, material is added (15A) until the implant 120 is large enough to occlude the lumen (15B). Bulking materials 120 may include any number of materials that are biocompatible with the surrounding body tissue. In one embodiment, the bulking material 120 may include a balloon, similar to that previously described. In another embodiment, the bulking material 120 may include a free-flowing material such as a carrier gel/matrix including beads or microspheres. In yet another embodiment, the bulking material 120 may include a paste, such as an auto-polymerizing paste (e.g., polymerizes upon injection and contact with the surrounding body tissue). In yet another embodiment, the bulking material 120 may include a biologically-based material, such as collagen or a hydroxyapatite-based material. The aforementioned bulking materials 120 can be injected and/or placed by any number of suitable delivery methods and devices known in the art, including, but not limited to catheter, needle and syringe delivery methods and devices.

Any of the implants 10 of the present invention may be inserted by numerous methods and/or in various locales in the uterine region of a female patient. Many of the embodiments described above are shaped to provide a degree of anchoring and stability in the interim period before ingrowth anchors the implant 10 in place. Additionally, as one skilled in the art will realize, the implants 10 of the present invention easily accept sutures for attachment to the target site.

An implant 10 may be inserted into the LUS and/or cervix, in a manner that will occlude the area in several ways. One example of such occlusion of the LUS and/or cervix is placement of the implant 10 in a manner sufficient to create an occlusion by exerting force outward upon the surrounding lumen tissue (radial exertion, as with aforementioned balloon 10).

Another example of such occlusion of the LUS and/or cervix is placement of the implant 10 in a manner sufficient to plug or “block” the LUS and/or cervical region. Several examples of blocking may include: placing an implant 10 in a manner sufficient to block the external os region of the cervix; placing an implant 10 in a manner sufficient to block the internal os region of the cervix; placing a implant 10 in a manner sufficient to block both the external and internal os regions of the cervix (e.g., as shown in FIG. 14); and/or placing an implant 10 in a manner sufficient to restrict menstrual flow (in this example, a small lumen may be placed through the implant 10, to release air and/or fluid from the uterus). In the example of the implant 10 having a lumen therein to restrict menstrual flow, it is hypothesized that such occlusion, although permitting some restricted flow from the uterus, still results in a significant portion of the endometrium becoming attenuated.

The aforementioned embodiments of occluding devices or implants 10 and methods for placement as described herein are further contemplated for use in or near the uterine cavity of a female patient, whether the uterine tissue layers (e.g., the endometrial layer, the myometrial layer) and/or epithelial layers at or near the uterus are intact or not.

One embodiment of a method for placing an implant in a lumen of a body cavity of a patient includes a tissue pre-treatment step. A pre-treatment step is beneficial to prepare the lumen tissues to receive the implant 10 and to facilitate tissue ingrowth into the implant. One method for preparing the lumen tissue for receiving an implant 10 is to impart damage to the tissue area where the implant 10 will be placed. One example of such tissue damage may include imparting thermal damage. Thermal damage may be accomplished using RF energy, resistive heating elements, ultrasound, lasers, etc. Typically, within the uterus, endometrial tissue is either first removed or allowed to remain, and then RF is applied to the surface tissue. The desired embodiment is such that only the surface of the myometrium has RF applied to it. Application in the cervix is slightly different in that there is no endometrium, thus the RF may be applied to the tissue surface. The RF is either applied while the implant 10 is in position in the cavity, or immediately prior to placing the implant.

The preceding paragraphs describing use of thermal energy to create tissue damage should not be construed as limiting. These occluding devices or implants 10 may be utilized without a thermal application, so long as the myometrial tissue layer of the uterus is exposed. For example, another embodiment of a method for preparing tissue for implantation of an occluding device or implant 10 may include a method in which all or part of the endometrium is removed mechanically. Tissue removal tools and methods may include such tools and methods that are known in the art, including scraping, scalpels and morcellators.

Two examples of scenarios regarding how to drive tissue contact with the implant include lumen constriction onto the implant 10, due to damage caused by pre-treatment, and implant 10 expansion into the tissue, which remains in its current spatial orientation and/or retracts. In the first example, once damaged (e.g. thermal energy), the lumen begins to constrict down onto the implant 10. This newly formed intimate contact fosters the tissue ingrowth into the implant 10. In this instance, implants 10 shaped to conform or take advantage of the uterine/cervical anatomy will foster the intimate contact. In the second example, the tissue maintains its current spatial position and an implant 10 is employed which expands to create intimate contact with the treated tissue. Examples of such expandable implants 10 may include balloon-based devices 10 as earlier described. Another example would be an oversized implant 10 that may be compressed into a delivery tool (e.g., delivery catheter, tube, syringe or needle) and, when deployed, would expand to its maximum size and contact the tissue walls. For example, in a preferred embodiment, an implant 10 may have an original maximum size of 6 mm in diameter and be compressible to 2 mm-4 mm diameter for placement within a delivery tool. When deployed into a lumen, the implant 10 would thereafter expand against the tissue walls to its original 6 mm diameter.

There are various methods of deployment by which an implant 10 may be introduced into a lumen of a body cavity. In general (and as shown in FIG. 16), implant delivery tools and deployment tools that are known in the medical arts are contemplated for use herein (e.g., delivery catheters, tubes, syringes and/or needles). FIGS. 16A-C illustrate one embodiment of a deployment system 140 for delivering an implant 10 into a lumen of body cavity. The delivery system 140 includes a delivery catheter 142, which houses a pusher tube 144 attached to an implant 10 as previously described. In this embodiment, the implant 10 is compressed into the delivery catheter 142 and ejected through the delivery catheter 142 by the pusher tube 144. The pusher tube 144 may hold the implant 10 in place by various methods, including but not limited to, vacuum and/or suction, hooks, adhesive, etc., depending upon the most optimal method for use with the particular implant 10. FIGS. 16A-C illustrate a sequence in which an implant 10 is being deployed into a prone cervix D. The catheter 142 is situated with its distal end 146 at or near the target site. The pusher tube 144 is advanced through the delivery catheter 42 until the implant 10 is delivered at the desired location in the cervix D. When the implant 10 is released from the delivery catheter 142, it is permitted to expand to its deployed state inside of the cervix D, thereby occluding the cervix D. Another embodiment comprises the same components, however the method of deployment is different. Upon situation of the catheter 142 at the target site, the pusher tube is held stationary, while the catheter tube 142 is withdrawn, exposing and consequently releasing the implant 10.

Additional delivery tools 40 that are known for use in insertion of IUDs or for inserting a morcellator (for imparting tissue damage prior to or during placing an implant, as previously described herein) are also contemplated for use herein in placing an implant. One preferred embodiment for delivering an implant 10 (not shown) is to provide a tube that houses the implant, the tube having a movable indicator ring thereon that is used to set the desired insertion distance (e.g., the distance from the body opening to the cervical os. This ring may be moved to the appropriate distance such that a physician knows how far to insert the tube into the patient's body. Thereafter, a piston may be used to deploy the implant into the lumen of a body cavity.

Although the invention has been described in terms of particular embodiments, methods and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-15. (canceled)

16. Method of occluding a cervix comprising:
inserting a foreign material into tissue surrounding a cervical lumen that places inward pressure on the walls of the cervical lumen, thereby closing the cervical lumen.

17. The method of claim 16 wherein inserting a foreign material comprises injecting a bulking agent.

18. The method of claim 16 wherein inserting a foreign material comprises implanting one or more balloons.

19. The method of claim 18 further comprising inflating said balloons until said cervical lumen is closed.

20. The method of claim 17 wherein said bulking agent comprises a liquid.

21-25. (canceled)

26. The method of claim 17 wherein said bulking agent comprises a gel matrix.

27. Method of occluding a cervix comprising:
deploying at least a portion of a foreign material into a cavity between an external cervical os and a fallopian tube that places outward pressure on a wall of the cavity, a portion of said foreign material being substantially cylindrical when deployed; and
anchoring the foreign material in the cavity by the outward pressure.

28. The method of claim 27 wherein deploying at least a portion of a foreign material into a cavity comprises deploying the foreign material in a lower uterine segment.

29. The method of claim 27 wherein deploying at least a portion of a foreign material into a cavity comprises deploying the foreign material in a cervical lumen.

30. The method of claim 27 wherein deploying at least a portion of a foreign material into a cavity comprises deploying the foreign material in an internal cervical os.

31. The method of claim 27 wherein deploying at least a portion of a foreign material into a cavity comprises implanting one or more balloons.

32. The method of claim 31 further comprising inflating the one or more balloons until the cervical lumen is closed.

33. Method of occluding a cervix comprising:
occupying a cross-sectional area of a cavity between a uterus and a vagina with a substantially cylindrical foreign material;
maintaining the substantially cylindrical foreign material in the cross-sectional area of the cavity; and
adhering said substantially cylindrical foreign material to at least a portion of a wall of the cross-sectional area of the cavity.

34. The method of claim 33 wherein occupying a cross-sectional area of a cavity between a uterus and a vagina with a substantially cylindrical foreign material comprises implanting a porous foreign material.

35. The method of claim 33 wherein occupying a cross-sectional area of a cavity between a uterus and a vagina with a substantially cylindrical foreign material comprises implanting the foreign material in a collapsed configuration.

36. The method of claim 35 further comprising transforming the substantially cylindrical foreign material from the collapsed configuration to an expanded configuration.

37. The method of claim 36 wherein transforming the substantially cylindrical foreign material from the collapsed configuration to an expanded configuration comprises transposing a fluid into the foreign material.

38. The method of claim 33 wherein occupying a cross-sectional area of a cavity between a uterus and a vagina with a substantially cylindrical foreign material comprises occupying a cross-sectional area of a lower uterine segment.

39. The method of claim 33 wherein occupying a cross-sectional area of a cavity between a uterus and a vagina with a substantially cylindrical foreign material comprises occupying a cross-sectional area of a cervical lumen.

40. The method of claim 33 wherein occupying a cross-sectional area of a cavity between a uterus and a vagina with a substantially cylindrical foreign material comprises occupying a cross-sectional area of a cervical os.

41. The method of claim 33 wherein adhering said substantially cylindrical foreign material to at least a portion of a wall of the cross-sectional area of the cavity comprises promoting ingrowth of a tissue in the cavity into the foreign material.
